SEQUENCE LISTING (7 sequences)

SEQ ID NO: 1 - protein, 35 aa, artificial
  synthetic
  DOMAIN (1)..(35): Module C (ADF-4)
GSSAAAAAAAASGPGGYGPENQGPSGPGGYGPGGP

SEQ ID NO: 2 - protein, 35 aa, artificial
  synthetic
  DOMAIN (1)..(35): Module Cc
GSSAAAAAAAASGPGGYGPENQGPCGPGGYGPGGP

SEQ ID NO: 3 - protein, 560 aa, artificial
  synthetic
  REPEAT (1)..(560): C16
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 4 - protein, 1120 aa, artificial
  synthetic
  REPEAT (1)..(1120): C32
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 5 - protein, 1680 aa, artificial
  synthetic
  REPEAT (1)..(1680): C48
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 6 - protein, 280 aa, artificial
  synthetic
  REPEAT (1)..(280): C8
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 7 - protein, 40 aa, Artificial
  synthetic
  Domain (1)..(40): Module Clys
GRGSMGSSAAAAAAAASGPGGYGPKNQGPSGPGGYGPGGP

(19) United States
(12) Patent Application Publication
(10) Pub. No.: US 2024/0279473 A1
(43) Pub. Date: Aug. 22, 2024

(54) SILK POLYPEPTIDE FORMULATION COMPRISING UREA

(71) Applicant: AMSILK GMBH, Planegg/Martinsried (DE)

(72) Inventors: Lin Romer, Ottobrunn (DE); Andreas Schmideder, Velden (DE); Ralf Mehrwald, Muenchen (DE)

(21) Appl. No.: 18/568,194

(22) PCT Filed: Jun. 2, 2022
(86) PCT No.: PCT/EP2022/065143

§ 371 Date: Dec. 7, 2023

(30) Foreign Application Priority Data
Jun. 10, 2021 (EP) 21178895.5

(51) Int. Cl. C08L 89/00; C07K 14/435

(52) U.S. Cl. CPC C08L 89/00; C07K 14/43518; C08L 2201/54

(57) ABSTRACT

The present invention relates to an aqueous formulation comprising a silk polypeptide and urea. The present invention further relates to a method for producing said formulation. The present invention also relates to uses of said formulation.

The present invention relates to an aqueous formulation comprising a silk polypeptide and urea. The present invention further relates to a method for producing said formulation. The present invention also relates to uses of said formulation.

### BACKGROUND OF THE INVENTION

Silk polypeptides represent a unique family of structural molecules. The molecular structure of many silks consists of large regions or domains of hydrophobic amino acids, segregated by relatively short and more hydrophilic regions. Silk polypeptides exhibit superior mechanical properties. They are promising materials for drug delivery and tissue engineering due to their biocompatibility, biodegradability, self-assembly, and controllable structure and morphology. Additionally, silk materials exhibit high encapsulation efficiency and controllable drug release kinetics due to the adjustment of crystalline-sheet formation. The formation of silk polypeptide biomaterials, such as fibers, films, mats, scaffolds, or capsules, is well known in the art. Moreover, silk polypeptides are well known in the cosmetic field, e.g. as basic compounds.

However, the known silk polypeptide solutions have the disadvantage that the silk polypeptides are not homogeneously distributed therein at high concentrations. Silk polypeptide precipitation at high concentrations is, however, detrimental to the quality of the resulting silk products.

Thus, there is still a need for new silk polypeptide formulations which can be used in many applications such as in the agrochemical industry, cleaning industry, home care industry, detergent industry, cosmetic industry, or food industry. These new silk polypeptide formulations should particularly comprise silk polypeptides at high concentrations. It would especially be advantageous for many applications, e.g. in the agrochemical industry, cleaning industry, home care industry, detergent industry, cosmetic industry, or food industry, to use liquid or flowable formulations which are easy to apply. These liquid or flowable formulations should comprise the highest possible concentrations of silk polypeptides to prevent dilution of the other active substances and to minimize the environmental footprint for transportation as well as the volumes to be used by the user. In addition, the production costs for silk polypeptide formulations should be significantly reduced in order to ensure a sustainable market entry, in particular in application areas with lower added value.

The present inventors provide new aqueous formulations comprising a silk polypeptide and urea. In addition, the present inventors provide a new production process for the generation of aqueous formulations comprising a silk polypeptide and urea. These formulations encompass silk polypeptides at high concentrations. In particular, the presence of fibrillary structures comprised in the silk polypeptide formulations simplifies and improves the coating of substrates with said formulations. Furthermore the use of urea as solvent has the advantage that it does not have to be removed subsequently. It does not adversely affect subsequent processes and applications. Accordingly, the new aqueous formulations can be used in different fields, e.g. in the agrochemical industry, cleaning industry, home care industry, detergent industry, cosmetic industry, or food industry.

### SUMMARY OF THE INVENTION

In a first aspect, the present invention relates to an aqueous formulation comprising a silk polypeptide and urea, wherein the concentration of the silk polypeptide in the formulation is in the range of 0.5% by weight to 30% by weight.

In a second aspect, the present invention relates to a method for producing an aqueous formulation comprising the steps of:
(i) providing an aqueous dispersion comprising a silk polypeptide and urea, wherein the concentration of the silk polypeptide in the dispersion is in the range of 0.5% by weight to 30% by weight, and
(ii) forming an aqueous formulation from the dispersion, wherein the silk polypeptide is comprised in a solubilized form.

In a third aspect, the present invention relates to an aqueous formulation obtainable by the method according to the second aspect.

In a fourth aspect, the present invention relates to the use of the aqueous formulation according to the first or third aspect for silk polypeptide storage.

In a firth aspect, the present invention relates to the use of the aqueous formulation according to the first or third aspect in the agrochemical industry, cleaning industry, detergent industry, home care industry, cosmetic industry, or food industry, preferably pet food industry.

In a sixth aspect, the present invention relates to a substrate comprising the aqueous formulation according to the first or third aspect.

In a seventh aspect, the present invention relates to a composition comprising the aqueous formulation according to the first or third aspect.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The terms “polypeptide” and “protein” are used interchangeably in the context of the present invention. They refer to a long peptide-linked chain of amino acids, e.g. one that is at least 20 amino acids long.

The term “silk polypeptide”, as used herein, refers to a polypeptide which shows, in comparison to other polypeptides, a quite aberrant amino acid composition. In particular, a silk polypeptide possesses large quantities of hydrophobic amino acids such as glycine or alanine. In addition, a silk polypeptide contains highly repetitive amino acid sequences or repetitive units (repeat units, modules), especially in their large core domain.

Based on DNA analysis, it was shown that all silk polypeptide are chains of repetitive units which further comprise a limited set of distinct shorter peptide motifs. The expressions “peptide motif” and “consensus sequence” can be used interchangeably herein. Generally, the silk consensus sequences can be grouped into four major categories: GPGXX, GGX, Aₓ or (GA)ₙ and spacers. These categories of peptide motifs in silk proteins have been assigned structural roles. For example, it has been suggested that the GPGXX motif is involved in a β-turn spiral, probably providing elasticity. The GGX motif is known to be responsible for a glycine-rich 3₁-helix. Both GPGXX and GGX motifs are thought to be involved in the formation of an amorphous matrix that connects crystalline regions, thereby providing elasticity of the fiber. Alanine-rich motifs typically contain 6-9 residues and have been found to form crystalline β-sheets. The spacers typically contain charged groups and separate the iterated peptide motifs into clusters. The silk polypeptide can perform self-assembly. Preferably, the silk polypeptide is a spider silk polypeptide. More preferably, the silk polypeptide, e.g. spider silk polypeptide, is a recombinant polypeptide.

The term “self-assembly”, as used herein, refers to a process in which a disordered system of pre-existing polypeptides forms an organized structure or pattern as a consequence of specific, local interactions (e.g. van der Waals forces, hydrophobic interactions, hydrogen bonds, and/or salt-bridges, etc.) among the polypeptides themselves, without external direction or trigger although external factors might influence speed and nature of self-assembly. This particularly means that when two or more disordered and/or unfolded polypeptides are brought into contact, they interact with each other and consequently form a three-dimensional structure. This three-dimensional structure can also be considered as a polypeptide aggregate. The change from a disordered system to an organized structure or pattern during self-assembly is characterized by a transition from a fluid state to a fibrillary or gelatinous/gel-like and/or solid state and a corresponding increase in viscosity. The transition from a fluid state to a gelatinous/gel-like and/or solid state can be monitored, for example, by optical measurement or rheology. These techniques are known to the skilled person. The transition from a fluid state to a gelatinous/gel-like and/or solid state can be monitored, for example, using optical methods. As mentioned above, polypeptide aggregates are formed during the process of self-assembly.

The term “aqueous dispersion”, as used herein, refers to any dispersion in which water (H₂O) is comprised. It is a two-phased system that is made up of extremely fine particles that are uniformly distributed throughout water. Under normal conditions, the solids would not be evenly distributed throughout the water. In the context of the present invention, the aqueous dispersion encompasses, in addition to water, urea and a silk polypeptide. The silk polypeptide is comprised in the aqueous dispersion of the present invention in non-solubilized or partially solubilized form. The aqueous dispersion can have a viscosity which is lower than the viscosity of the aqueous formulation (described below). In this case, the aqueous formulation is present in gel or solid form. The aqueous dispersion has preferably a pH of <9, e.g. a pH of <5, 6, 7, 8, or 9. The present inventors surprisingly found that an aqueous dispersion comprising urea and a silk polypeptide can be transferred or converted into an aqueous formulation such as aqueous solution by adding a base or a buffer comprising a base. Thereby, the pH of the aqueous dispersion is increased, e.g. to a pH ≥9, resulting in the solubilization of the silk polypeptide.

The term “aqueous solution”, as used wherein, refers to any solution in which water (H₂O) is the solvent. Although water is often called the universal solvent, it dissolves only substances that are hydrophilic in nature such as acids, bases and salts. The aqueous solution of these items mixes together with the water completely. Hydrophobic items do not dissolve very well in water, such as oils and fats. In the context of the present invention, the aqueous solution encompasses, in addition to water, urea and a silk polypeptide. The silk polypeptide is comprised in the aqueous solution of the present invention in solubilized form. The aqueous solution can have a viscosity which is lower than the viscosity of the aqueous formulation (described below). In this case, the aqueous formulation is present in gel or solid form.

As mentioned above, the present inventors surprisingly found that an aqueous dispersion comprising urea and a silk polypeptide can be transferred or converted into an aqueous formulation such as aqueous solution by adding a base or a buffer comprising a base. The aqueous solution may contain remnants of dispersed protein.

The aqueous dispersion has preferably a pH of <9, e.g. a pH of <5, 6, 7, 8, or 9. Due to the addition of a base or a buffer comprising a base, the pH of the aqueous dispersion is increased, e.g. to a pH ≥9, resulting in the solubilization of the silk polypeptide. The pH of the aqueous dispersion is preferably increased to a pH in the range of 9 to 13, more preferably to a pH in the range of 11.5 to 13, and even more preferably to a pH in the range of 12 to 13. In this way, an aqueous formulation such as aqueous solution is produced. In order to be able to use the aqueous formulation such as aqueous solution comprising urea and a silk polypeptide for as many applications as possible, it is practical to (subsequently) lower the pH (again), e.g. to a pH <9, by adding an acid buffer or an acid to said aqueous formulation such as aqueous solution. In this way, the base comprised in the aqueous formulation such as aqueous solution comprising urea and a silk polypeptide is neutralized. The final pH of the aqueous formulation such as aqueous solution comprising urea and a silk polypeptide after neutralization is preferably in the range of 5.5 to 11, more preferably in the range of 6.5 to 11, even more preferably in the range of pH 7 to 10, and still even more preferably in the range of 8 to 9.

The term “aqueous formulation”, as used herein, refers to an aqueous formulation comprising urea and a silk polypeptide in solubilized form. Within the aqueous formulation, the silk polypeptide is preferably homogenously distributed. The aqueous formulation is preferably an aqueous solution. The aqueous formulation can also have a viscosity which is higher than the viscosity of an aqueous solution. In this case, the aqueous formulation is present in gel or solid form.

The term “compound”, as used herein, refers to any compound having a purpose that may be useful in the present invention, e.g. a compound useful in the agrochemical industry, cleaning industry, home care industry, detergent industry, cosmetic industry, or food industry. The compound is preferably part of the aqueous formulation such as aqueous solution described herein. Aqueous formulations comprising the compound may be formulated by mixing the compound with the aqueous dispersion used to make the aqueous formulation. Alternatively, the compound can be coated onto, attached to, or incorporated in the aqueous formulation after its formation. For example, the compound can be added to the aqueous solution which is finally formed. The compound may be present as a liquid, a finely divided solid or in any other appropriate physical form. The compound useful in the agrochemical industry may be selected from the group consisting of dyestuff, odoriferous substance, sunscreen, fertilizer, pesticide, hormone, growth factor, insecticide, pesticide, herbicide, fungizide, microorganisms, and nutrient. The compound useful in the food industry may be selected from the group consisting of a nutrient, vitamin, aroma, preservative and dye. The compound useful in the cleaning industry and homecare industry may be selected from the group consisting of dyestuff, antimicrobial substance, antiviral substance, detergent, softener, surfactant, enzyme, fragrance, alcohol, greying inhibitor, dye transfer inhibitor, metal complex, nitrile, inorganic compound, bleaching agent, tenside, fatty acid, carbonic acid, silicate, carbonate, polymer, silicone, and brightening agent.

The aqueous formulation described herein may alternatively be part of a substrate or composition. The substrate or composition may be, for example, a substrate or composition useful in the agrochemical industry, cleaning industry, home care industry, detergent industry, cosmetic industry, or food industry. Such composition may be a food composition, a cleaning composition, a fertilizer composition, or a plant or seed protecting composition. In these cases, the aqueous formulation may be integrated, embedded, or worked into the composition.

The aqueous formulation described herein may be applied as a coating. For example, a substrate may be coated with the aqueous formulation.

The term “coating”, as used herein, refers to a covering that is applied to the substrate, in particular to the surface of the substrate, to be coated. The coating itself may be an all-over coating, completely covering the substrate, or it may only cover parts of the substrate.

In one embodiment, the coating covers at least 1%, preferably at least 30%, more preferably at least 50%, even more preferably at least 80%, and most preferably at least 90% or even 100% of the surface of the substrate. In one preferred embodiment, the coating is an uniform and/or homogenous coating. It has preferably a thickness of between 10 nm and 1 mm and more preferably a thickness of between 50 nm and 0.5 μm.
The coating is preferably achieved by dip coating and/or spray coating.
In an example, the substrate is a plant. In this case, the coating may be applied to (parts of) the root(s) of the plant and/or sprout(s) of the plant/aboveground part(s) of the plant. The coating may also be applied to (parts of) the fruits and/or blossoms/flower petals of the plant. It should be clear that a plant, in particular (a/an) sprout(s) of a plant/aboveground part(s) of a plant, also comprise(s) leaves/leafage. Thus, the coating preferably also covers the leaves/leafage. The coating is preferably applied pre-harvest, e.g. to a seedling, a growing plant, or full-grown plant.

The aqueous formulation described herein may alternatively be part of a composition. The composition may be, for example, a composition useful in the agrochemical industry, cleaning industry, home care industry, detergent industry, cosmetic industry, or food industry. Such composition may be a food composition, a cleaning composition, a fertilizer composition, or a plant or seed protecting composition. In these cases, the aqueous formulation may be integrated, embedded, or worked into the composition.

### EMBODIMENTS OF THE INVENTION

The present inventors provide new aqueous formulations comprising a silk polypeptide and urea. In addition, the present inventors provide a new production process for the generation of aqueous formulations comprising a silk polypeptide and urea. These formulations encompass silk polypeptides at high concentrations. The use of urea as solvent has the advantage that it does not have to be removed subsequently. It does not adversely affect subsequent processes and applications. Accordingly, the new aqueous formulations can be used in different fields, e.g. in the agrochemical industry, cleaning industry, home care industry, detergent industry, cosmetic industry, or food industry.

Thus, in a first aspect, the present invention relates to an aqueous formulation comprising a silk polypeptide and urea, wherein the concentration of the silk polypeptide in the formulation is in the range of 0.5% by weight to 30% by weight, e.g. 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30% by weight.

In one preferred embodiment, the aqueous formulation is liquid, i.e. is an aqueous solution, has a fibrillary or gel-like structure, or is solid. The fibrillary or gel-like structure may be a hydrogel. It is particularly preferred that the aqueous formulation is an aqueous solution

Preferably, the concentration of the silk polypeptide in the aqueous formulation such as aqueous solution is in the range of 0.5% by weight to 15% by weight, more preferably in the range of 2% by weight to 15% by weight, even more preferably in the range of 4% by weight to 15% by weight, and still even more preferably in the range of 5% by weight to 15% by weight, e.g. 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15% by weight.

It is particularly preferred that the concentration of the silk polypeptide in this formulation is at least 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5% by weight, or 30% by weight. It is more particularly preferred that the concentration of the silk polypeptide in this formulation is at least 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5% by weight and not more than 30% by weight.

Preferably, the concentration of urea in the aqueous formulation is in the range of 3 M to 10 M, more preferably in the range of 5 M to 10 M, and even more preferably in the range of 8 M and 10 M, e.g. 3, 4, 5, 6, 7, 8, 9, or 10 M. Usually, a urea stock solution of between 5 and 10 M, e.g. of 5, 8, or 10 M, is used.

Thus, in one more preferred embodiment, the aqueous formulation such as aqueous solution comprises a silk polypeptide and urea, wherein

the concentration of the silk polypeptide in the aqueous formulation is in the range of 0.5% by weight to 30% by weight, e.g. 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30% by weight, and
the concentration of urea in the aqueous formulation is in the range of 3 M to 10 M, e.g. 3, 4, 5, 6, 7, 8, 9, or 10 M.

In one even more preferred embodiment, the aqueous formulation such as aqueous solution comprises a silk polypeptide and urea, wherein

the concentration of the silk polypeptide in the aqueous formulation is in the range of 0.5% by weight to 15% by weight, e.g. 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15% by weight, and
the concentration of urea in the aqueous formulation is in the range of 3 M to 10 M, e.g. 3, 4, 5, 6, 7, 8, 9, or 10 M.

In one still even more preferred embodiment, the aqueous formulation such as aqueous solution comprises a silk polypeptide and urea, wherein

the concentration of the silk polypeptide in the aqueous formulation is in the range of 5% by weight to 15% by weight, e.g. 5, 5.5, 6, 6.5, 7, 7.5, 8, 8.5, 9, 9.5, 10, 10.5, 11, 11.5, 12, 12.5, 13, 13.5, 14, 14.5, or 15% by weight, and
the concentration of urea in the aqueous formulation is in the range of 8 M to 10 M, e.g. 8, 9, or 10 M.

The present inventors surprisingly found that an aqueous dispersion comprising urea and a silk polypeptide can be transferred or converted into an aqueous formulation such as aqueous solution by adding a base or a buffer comprising a base. Preferably, the aqueous dispersion has a pH of <9, e.g. a pH of <5, 5.5, 6, 6.5, 7, 7.5, 8, 8.5, or 9. Due to the addition of a base or buffer comprising a base, the pH of the aqueous dispersion is increased, e.g. to a pH ≥9, resulting in the solubilization of the silk polypeptide. The pH of the aqueous dispersion is preferably increased to a pH in the range of 9 to 13, more preferably to a pH in the range of 11.5 to 13, and even more preferably to a pH in the range of 12.5 to 13, e.g. to a pH of 9, 9.5, 10, 10.5, 11, 11.5, 12, 12.5, or 13.

Thus, the aqueous formulation such as aqueous solution has preferably a pH in the range of 9 to 13, more preferably in the range of 11.5 to 13, and even more preferably in the range of 12 to 13, e.g. a pH of 9, 9.5, 10, 10.5, 11, 11.5, 12, 12.5, or 13 (before neutralization).

In order to be able to use the aqueous formulation such as aqueous solution comprising urea and a silk polypeptide for as many applications as possible and to avoid unwanted hydrolyzation over time, it is practical to (subsequently) lower the pH (again), e.g. to a pH <9, by adding an acid buffer or an acid to said aqueous formulation such as aqueous solution. In this way, the base comprised in the aqueous formulation such as aqueous solution comprising urea and a silk polypeptide is neutralized.

In this case, the aqueous formulation such as aqueous solution has preferably a pH in the range of 5.5 to 11, more preferably a pH in the range of 6.5 to 11, even more preferably a pH in the range of pH 7 to 10, and still even more preferably a pH in the range of 8 to 9, e.g. a pH of 5.5, 6, 6.5, 7, 7.5, 8, 8.5, 9, 9.5, 10, 10.5, or 11 (after neutralization).

It is (alternatively or additionally) preferred that the silk polypeptide is a recombinant silk polypeptide. The (recombinant) silk polypeptide may be a spider silk polypeptide, e.g. a major ampullate silk polypeptide such as a dragline silk polypeptide, a minor ampullate silk polypeptide, or a flagelliform silk polypeptide of an orb-web spider. Particularly, the silk polypeptide is a spider silk polypeptide. More particularly, the spider silk polypeptide is a recombinant spider silk polypeptide.

It is particularly preferred that the (recombinant) silk polypeptide is a polypeptide with an amino acid sequence which comprises or consists of at least 50%, 60%, 65%, 70%, 75%, 80%, 85%, or 90% multiple copies of repetitive units. It is more preferred that the silk polypeptide is a polypeptide with an amino acid sequence which comprises or consists of at least 95% multiple copies of repetitive units. Said repetitive units may be identical or different.
It is further particularly preferred that the (recombinant) silk polypeptide consists of between 40 to 4000 amino acids. It is more preferred that the (recombinant) silk polypeptide consists of between 100 to 3500 amino acids or between 200 to 2500 amino acids. It is even more preferred that the (recombinant) silk polypeptide consists of between 250 to 2000 amino acids.
It is also particularly preferred that the (recombinant) silk polypeptide comprises at least two identical repetitive units. For example, the (recombinant) silk polypeptide may comprise between 2 to 100 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 repetitive units.
It is particularly more preferred that the repetitive units are independently selected from the group consisting of module C (SEQ ID NO: 1) or variants thereof, module C^{Cys} (SEQ ID NO: 2), and module C^{Lys} (SEQ ID NO: 7). Module C^{Cys} (SEQ ID NO: 2) is a variant of module C (SEQ ID NO: 1). In this module, the amino acid Ser at position 25 has been replaced by the amino acid Cys. Module C^{Lys} (SEQ ID NO: 7) is also a variant of module C (SEQ ID NO: 1). Module C^{Cys} can also be designated as module CC.
The module C variant differs from the reference module C from which it is derived by up to 1, 2, 3, 4, 5, 6, 7, 8, 9, or 10, 11, 12, 13, 14, or 15 amino acid changes in the amino acid sequence (i.e. substitutions, additions, insertions, deletions, N-terminal truncations and/or C-terminal truncations). Such a module variant can alternatively or additionally be characterized by a certain degree of sequence identity to the reference module from which it is derived. Thus, the module C variant has a sequence identity of at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even 99.9% to the respective reference module C. Preferably, the sequence identity is over a continuous stretch of at least 5, 10, 15, 18, 20, 24, 27, 28, 30, 34, or more amino acids, preferably over the whole length of the respective reference module C.
The sequence identity may be at least 80% over the whole length, may be at least 85% over the whole length, may be at least 90% over the whole length, may be at least 95% over the whole length, may be at least 98% over the whole length, or may be at least 99% over the whole length of the respective reference module C. Alternatively, the sequence identity may be at least 80% over a continuous stretch of at least 5, 10, 15, 18, 20, 24, 28, or 30 amino acids, may be at least 85% over a continuous stretch of at least 5, 10, 15, 18, 20, 24, 28, or 30 amino acids, may be at least 90% over a continuous stretch of at least 5, 10, 15, 18, 20, 24, 28, or 30 amino acids, may be at least 95% over a continuous stretch of at least 5, 10, 15, 18, 20, 24, 28, or 30 amino acids, may be at least 98% over a continuous stretch of at least 5, 10, 15, 18, 20, 24, 28, or 30 amino acids, or may be at least 99% over a continuous stretch of at least 5, 10, 15, 18, 20, 24, 28, or 30 amino acids of the respective reference module C.
A fragment (or deletion) variant of module C has preferably a deletion of up to 1, 2, 3, 4, 5, 6, 7, 8, 9, 10, 11, 12, 13, 14, or 15 amino acids at its N-terminus and/or at its C-terminus. The deletion can also be internally.
Additionally, the module C variant or fragment is only regarded as a module C variant or fragment within the context of the present invention, if the modifications with respect to the amino acid sequence on which the variant or fragment is based do not negatively affect the ability of the silk polypeptide, comprised in an aqueous dispersion comprising urea, to be solubilized (without the formation of precipitate) due to the addition of a base or a buffer comprising a base. The skilled person can readily assess whether the silk polypeptide comprising a module C variant or fragment still has this property. In this respect, it is referred to the examples comprised in the experimental part of the present patent application. Module C^{Cys} or C^{Lys} variants may also be encompassed by the present invention. Regarding the module C^{Cys} or C^{Lys} variants, the same explanations/definitions apply which have been made with respect to the module C variant (see above).
It is particularly even more preferred that the silk polypeptide is selected from the group consisting of (C)ₘ, (C^{Cys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96.
It is particularly most preferred that the silk polypeptide is selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₄₈, C₁₆C^{Cys}, C₃₂C^{Cys}, and C₄₈C^{Cys}.
The silk polypeptide C₁₆ (16 times module C) has the amino acid sequence according to SEQ ID NO: 3, the silk polypeptide C₃₂ (32 times module C) has the amino acid sequence according to SEQ ID NO: 4, the silk polypeptide C₄₈ (48 times module C) has the amino acid sequence according to SEQ ID NO: 5, and the silk polypeptide C& has the amino acid sequence according to SEQ ID NO: 6 (8 times module C).

The aqueous formulation according to the first aspect may further comprise at least one compound. Said compound may be any compound having a purpose that may be useful in the present invention, e.g. a compound useful in the agrochemical industry, cleaning industry, home care industry, detergent industry, cosmetic industry, or food industry.

In a second aspect, the present invention relates to a method for producing an aqueous formulation comprising the steps of:
(i) providing an aqueous dispersion comprising a silk polypeptide and urea, wherein the concentration of the silk polypeptide in the dispersion is in the range of 0.5% by weight to 30% by weight, e.g. 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30% by weight, and
(ii) forming an aqueous formulation from the dispersion, wherein the silk polypeptide is comprised in a solubilized form.

The aqueous dispersion can be produced by adding the silk polypeptide into an aqueous solution. The silk polypeptide, when added into the aqueous solution, may be present as a finely divided solid (in powder form).

In one preferred embodiment, the aqueous formulation is liquid, i.e. is an aqueous solution, has a fibrillary or gel-like structure, or is solid. The fibrillary or gel-like structure may be a hydrogel. It is particularly preferred that the aqueous formulation is an aqueous solution.

Preferably, the concentration of the silk polypeptide in the aqueous dispersion is in the range of 0.5% by weight to 15% by weight, more preferably in the range of 2% by weight to 15% by weight, even more preferably in the range of 4% by weight to 15% by weight, and still even more preferably in the range of 5% by weight to 15% by weight, e.g. 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15% by weight.

It is particularly preferred that the concentration of the silk polypeptide in this dispersion is at least 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5% by weight, or 30% by weight. It is more particularly preferred that the concentration of the silk polypeptide in this dispersion is at least 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5% by weight and not more than 30% by weight.

Preferably, the concentration of urea in the aqueous dispersion is in the range of 3 M to 10 M, more preferably in the range of 5 M to 10 M, and even more preferably in the range of 8 M and 10 M, e.g. 3, 4, 5, 6, 7, 8, 9, or 10 M. Usually, a urea stock solution of between 5 and 10 M, e.g. 5, 8, or 10 M, is used.

Thus, in one more preferred embodiment, the aqueous dispersion comprises a silk polypeptide and urea, wherein

the concentration of the silk polypeptide in the aqueous dispersion is in the range of 0.5% by weight to 30% by weight, e.g. 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, or 30% by weight, and the concentration of urea in the aqueous dispersion is in the range of 3 M to 10 M, e.g. 3, 4, 5, 6, 7, 8, 9, or 10 M.

In one even more preferred embodiment, the aqueous dispersion comprises a silk polypeptide and urea, wherein

the concentration of the silk polypeptide in the aqueous formulation is in the range of 0.5% by weight to 15% by weight, e.g. 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15% by weight, and the concentration of urea in the aqueous formulation is in the range of 3 M to 10 M, e.g. 3, 4, 5, 6, 7, 8, 9, or 10 M.

In one still even more preferred embodiment, the aqueous dispersion comprises a silk polypeptide and urea, wherein

the concentration of the silk polypeptide in the aqueous dispersion is in the range of 5% by weight to 15% by weight, e.g. 5, 5.5, 6, 6.5, 7, 7.5, 8, 8.5, 9, 9.5, 10, 10.5, 11, 11.5, 12, 12.5, 13, 13.5, 14, 14.5, or 15% by weight, and
the concentration of urea in the aqueous dispersion is in the range of 8 M to 10 M, e.g. 8, 9, or 10 M.

Preferably, the aqueous dispersion has a pH of <9, e.g. a pH of <5, 5.5, 6, 6.5, 7, 7.5, 8, 8.5, or 9.

The present inventors surprisingly found that the aqueous dispersion comprising urea and a silk polypeptide can be transferred or converted into an aqueous formulation such as aqueous solution by increasing the pH of the aqueous dispersion.

Thus, in one preferred embodiment, the aqueous formulation is formed by increasing the pH, to a pH ≥9, of the aqueous dispersion. The pH of the aqueous dispersion is preferably increased to a pH in the range of 9 to 13, more preferably to a pH in the range of 11.5 to 13, and even more preferably to a pH in the range of 12 to 13, e.g. to a pH of 9, 9.5, 10, 10.5, 11, 11.5, 12, 12.5, or 13 (before neutralization). Thereby, an aqueous formulation preferably having a pH in the range of 9 to 13, more preferably a pH in the range of 11.5 to 13, even more preferably a pH in the range of 12 to 13, e.g. a pH of 9, 9.5, 10, 10.5, 11, 11.5, 12, 12.5, or 13 (before neutralization), is obtained. The pH increase results in/leads to the solubilization of the silk polypeptide.

In one more preferred embodiment, the pH of the aqueous dispersion is increased by adding a basic buffer or a base to said aqueous dispersion. The base may be selected from the group consisting of sodium hydroxide (NaOH) or potassium hydroxide (KOH).

In order to be able to use the aqueous formulation such as aqueous solution comprising urea and a silk polypeptide for as many applications as possible, the present inventors found that it is practical to (subsequently) lower the pH (again).

Thus, in one even more preferred embodiment, the method further comprises the step of reducing the pH, e.g. to a pH <9, of the aqueous formulation (again). The pH of the aqueous formulation such as aqueous solution is reduced preferably to a pH in the range of 5.5 to 11, more preferably to a pH in the range of pH 6.5 and 11, even more preferably to a pH in the range of pH 7 to 10, and still even more preferably to a pH in the range of 8 to 9, e.g. to a pH of 5.5, 6, 6.5, 7, 7.5, 8, 8.5, 9, 9.5, 10, 10.5, or 11 (after neutralization). Thereby an aqueous formulation such as aqueous solution having preferably a pH in the range of 5.5 to 11, more preferably a pH in the range of 6.5 and 11, even more preferably a pH in the range of 7 to 10, and still more preferably a pH in the range of 8 to 9, e.g. a pH of 5.5, 6, 6.5, 7, 7.5, 8, 8.5, 9, 9.5, 10, 10.5, or 11 (after neutralization), is obtained.
In one still even more preferred embodiment, the pH of the aqueous formulation such as aqueous solution is reduced by adding an acid buffer or an acid to said aqueous formulation such as aqueous solution. In this way, the base comprised in the aqueous formulation such as aqueous solution comprising urea and a silk polypeptide is neutralized. The acid may be selected from the group consisting of hydrochloric acid (HCl) or citric acid.

The silk polypeptide is preferably homogenously distributed in the aqueous formulation such as aqueous solution produced with the above method. The silk polypeptide can easily be stored therein, e.g. up to 1, 2, 3, 4, 5, 6 day(s), 1, 2, 3 week(s), 1, 2, 3, 4, 5, 6, 7, 8, 9, 10, 11 month(s), 1 or 2 year(s).

It is (alternatively or additionally) preferred that the silk polypeptide is a recombinant silk polypeptide. The (recombinant) silk polypeptide may be a spider silk polypeptide, e.g. a major ampullate silk polypeptide such as a dragline silk polypeptide, a minor ampullate silk polypeptide, or a flagelliform silk polypeptide of an orb-web spider. Particularly, the silk polypeptide is a spider silk polypeptide. More particularly, the spider silk polypeptide is a recombinant spider silk polypeptide.

It is particularly preferred that the (recombinant) silk polypeptide is a polypeptide with an amino acid sequence which comprises or consists of at least 50%, 60%, 65%, 70%, 75%, 80%, 85%, or 90% multiple copies of repetitive units. It is more preferred that the silk polypeptide is a polypeptide with an amino acid sequence which comprises or consists of at least 95% multiple copies of repetitive units. Said repetitive units may be identical or different.
It is further particularly preferred that the (recombinant) silk polypeptide consists of between 40 to 4000 amino acids. It is more preferred that the (recombinant) silk polypeptide consists of between 100 to 3500 amino acids or between 200 to 2500 amino acids. It is even more preferred that the (recombinant) silk polypeptide consists of between 250 to 2000 amino acids.
It is also particularly preferred that the (recombinant) silk polypeptide comprises at least two identical repetitive units. For example, the (recombinant) silk polypeptide may comprise between 2 to 100 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 repetitive units.
It is particularly more preferred that the repetitive units are independently selected from the group consisting of module C (SEQ ID NO: 1) or variants thereof, module C^{Cys} (SEQ ID NO: 2), and module C^{Lys} (SEQ ID NO: 7). Module C^{Cys} (SEQ ID NO: 2) is a variant of module C (SEQ ID NO: 1). In this module, the amino acid Ser at position 25 has been replaced by the amino acid Cys. Module C^{Lys} (SEQ ID NO: 7) is also a variant of module C (SEQ ID NO: 1). Module C^{Cys} can also be designated as module CC.
The module C variant differs from the reference module C from which it is derived by up to 1, 2, 3, 4, 5, 6, 7, 8, 9, or 10, 11, 12, 13, 14, or 15 amino acid changes in the amino acid sequence (i.e. substitutions, additions, insertions, deletions, N-terminal truncations and/or C-terminal truncations). Such a module variant can alternatively or additionally be characterized by a certain degree of sequence identity to the reference module from which it is derived. Thus, the module C variant has a sequence identity of at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even 99.9% to the respective reference module C. Preferably, the sequence identity is over a continuous stretch of at least 5, 10, 15, 18, 20, 24, 27, 28, 30, 34, or more amino acids, preferably over the whole length of the respective reference module C.
The sequence identity may be at least 80% over the whole length, may be at least 85% over the whole length, may be at least 90% over the whole length, may be at least 95% over the whole length, may be at least 98% over the whole length, or may be at least 99% over the whole length of the respective reference module C. Alternatively, the sequence identity may be at least 80% over a continuous stretch of at least 5, 10, 15, 18, 20, 24, 28, or 30 amino acids, may be at least 85% over a continuous stretch of at least 5, 10, 15, 18, 20, 24, 28, or 30 amino acids, may be at least 90% over a continuous stretch of at least 5, 10, 15, 18, 20, 24, 28, or 30 amino acids, may be at least 95% over a continuous stretch of at least 5, 10, 15, 18, 20, 24, 28, or 30 amino acids, may be at least 98% over a continuous stretch of at least 5, 10, 15, 18, 20, 24, 28, or 30 amino acids, or may be at least 99% over a continuous stretch of at least 5, 10, 15, 18, 20, 24, 28, or 30 amino acids of the respective reference module C.
A fragment (or deletion) variant of module C has preferably a deletion of up to 1, 2, 3, 4, 5, 6, 7, 8, 9, 10, 11, 12, 13, 14, or 15 amino acids at its N-terminus and/or at its C-terminus. The deletion can also be internally.
Additionally, the module C variant or fragment is only regarded as a module C variant or fragment within the context of the present invention, if the modifications with respect to the amino acid sequence on which the variant or fragment is based do not negatively affect the ability of the silk polypeptide, comprised in an aqueous dispersion comprising urea, to be solubilized (without the formation of precipitate) due to the addition of a base or a buffer comprising a base. The skilled person can readily assess whether the silk polypeptide comprising a module C variant or fragment still has this property. In this respect, it is referred to the examples comprised in the experimental part of the present patent application. Module C^{Cys} or C^{Lys} variants may also be encompassed by the present invention. Regarding the module C^{Cys} or C^{Lys} variants, the same explanations/definitions apply which have been made with respect to the module C variant (see above).
It is particularly even more preferred that the silk polypeptide is selected from the group consisting of (C)ₘ, (C^{Cys})ₘ, (C)ₘC^{Cys}, C^{Cys}(C)ₘ, C^{Lys}(C)ₘ, and (C)ₘC^{Lys}, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96.
It is particularly most preferred that the silk polypeptide is selected from the group consisting of C^{Lys}C₁₆, C^{Lys}C₃₂, C^{Lys}C₄₈, C₁₆C^{Lys}, C₃₂C^{Lys}, C₄₈C^{Lys}, C₁₆, C₃₂, C₄₈, C^{Cys}C₁₆, C^{Cys}C₃₂, C^{Cys}C₄₈, C₁₆C^{Cys}, C₃₂C^{Cys}, and C₄₈C^{Cys}.
The silk polypeptide C₁₆ (16 times module C) has the amino acid sequence according to SEQ ID NO: 3, the silk polypeptide C₃₂ (32 times module C) has the amino acid sequence according to SEQ ID NO: 4, the silk polypeptide C₄₈ (48 times module C) has the amino acid sequence according to SEQ ID NO: 5, and the silk polypeptide C₈ has the amino acid sequence according to SEQ ID NO: 6 (8 times module C).

In one particularly preferred embodiment, the present invention relates to a method for producing an aqueous formulation such as aqueous solution comprising the steps of:
(i) providing an aqueous dispersion comprising a silk polypeptide and urea, wherein the concentration of the silk polypeptide in the dispersion is in the range of 0.5% by weight to 30% by weight, specifically 0.5% by weight to 15% by weight, wherein the concentration of urea in the formulation is in the range of 5 M to 10 M, and wherein the aqueous dispersion has a pH of <9, and
(ii) forming an aqueous formulation such as aqueous solution from the aqueous dispersion, wherein the silk polypeptide is comprised in a solubilized form, by increasing the pH of the aqueous dispersion to a pH ≥9 due to the addition of a basic buffer or a base to the aqueous dispersion.
   The base may be selected from the group consisting of sodium hydroxide (NaOH) or potassium hydroxide (KOH).
   As to the preferred concentration of the silk polypeptide and urea, it is referred to the embodiments described above.

In one particularly more preferred embodiment, the present invention relates to a method for producing an aqueous formulation such as aqueous solution comprising the steps of:
(i) providing an aqueous dispersion comprising a silk polypeptide and urea, wherein the concentration of the silk polypeptide in the dispersion is in the range of 0.5% by weight to 30% by weight, specifically 0.5% by weight to 15% by weight, wherein the concentration of urea in the formulation is in the range of 5 M to 10 M, and wherein the aqueous dispersion has a pH of <9, and
(ii) forming an aqueous formulation such as aqueous solution from the aqueous dispersion, wherein the silk polypeptide is comprised in a solubilized form, by increasing the pH of the aqueous dispersion to a pH in the range of 9 to 13 due to the addition of a basic buffer or a base to the aqueous dispersion.
   The base may be selected from the group consisting of sodium hydroxide (NaOH) or potassium hydroxide (KOH).
   As to the preferred concentration of the silk polypeptide and the urea, it is referred to the embodiments described above.

In one particularly even more preferred embodiment, the present invention relates to a method for producing an aqueous formulation such as aqueous solution comprising the steps of:
(i) providing an aqueous dispersion comprising a silk polypeptide and urea, wherein the concentration of the silk polypeptide in the dispersion is in the range of 0.5% by weight to 15% by weight, specifically 0.5% by weight to 15% by weight, wherein the concentration of urea in the formulation is in the range of 5 M to 10 M, and wherein the aqueous dispersion has a pH of <9,
(ii) forming an aqueous formulation such as aqueous solution from the aqueous dispersion, wherein the silk polypeptide is comprised in a solubilized form, by increasing the pH of the aqueous dispersion to a pH ≥9 due to the addition of a basic buffer or a base to the aqueous dispersion, and
(iii) (subsequently) reducing the pH of the aqueous formulation such as aqueous solution (again) to a pH <9 by adding an acid buffer or an acid to said aqueous formulation such as aqueous solution.
   The base may be selected from the group consisting of sodium hydroxide (NaOH) or potassium hydroxide (KOH).
   The acid may be selected from the group consisting of hydrochloric acid (HCl) or citric acid. As to the preferred concentration of the silk polypeptide and the urea, it is referred to the embodiments described above.

In one particularly still even more preferred embodiment, the present invention relates to a method for producing an aqueous formulation such as aqueous solution comprising the steps of:
(i) providing an aqueous dispersion comprising a silk polypeptide and urea, wherein the concentration of the silk polypeptide in the dispersion is in the range of 0.5% by weight to 15% by weight, specifically 0.5% by weight to 15% by weight, wherein the concentration of urea in the formulation is in the range of 5 M to 10 M, and wherein the aqueous dispersion has a pH of <9, e.g. <7, 8, or 9,
(ii) forming an aqueous formulation such as aqueous solution from the aqueous dispersion, wherein the silk polypeptide is comprised in a solubilized form, by increasing the pH of the aqueous dispersion to a pH in the range of 9 to 13 due to the addition of a basic buffer or a base to the aqueous dispersion, and
(iii) (subsequently) reducing the pH of the aqueous formulation such as aqueous solution (again) to a pH in the range of 5.5 to 11 by adding an acid buffer or an acid to said aqueous formulation.
   The base may be selected from the group consisting of sodium hydroxide (NaOH) or potassium hydroxide (KOH).
   The acid may be selected from the group consisting of hydrochloric acid (HCl) or citric acid. As to the preferred concentration of the silk polypeptide and the urea, it is referred to the embodiments described above.

The aqueous formulation according to the first aspect may further comprise at least one compound. Said compound may be any compound having a purpose that may be useful in the present invention, e.g. a compound useful in the agrochemical industry, cleaning industry, home care industry, detergent industry, cosmetic industry, or food industry.

Aqueous formulations comprising the compound may be formulated/produced by mixing the compound with the aqueous dispersion used to make the aqueous formulation. Alternatively, the compound can be coated onto, attached to, or incorporated in the aqueous formulation after its formation. For example, the compound can be added to the aqueous formulation such as aqueous solution which is finally formed. The compound may be present as a liquid, a finely divided solid or in any other appropriate physical form.

In a third aspect, the present invention relates to an aqueous formulation such as aqueous solution obtainable by the method according to the second aspect.

In a fourth aspect, the present invention relates to an aqueous formulation such as aqueous solution according to the first or third aspect for silk polypeptide storage.

The silk polypeptide can easily be stored therein, e.g. up to 1, 2, 3, 4, 5, 6 day(s), 1, 2, 3 week(s), 1, 2, 3, 4, 5, 6, 7, 8, 9, 10, 11 month(s), 1 or 2 year(s).

The formulation according to the first or third aspect can be used universally for all applications. In contrast to the previous production process for silk solutions, expensive solvents and complex procedures can be dispensed with. Thus, the manufacturing costs can be significantly reduced.

Thus, in a fifth aspect, the present invention relates to the use of the aqueous formulation such as aqueous solution according to the first or third aspect in the agrochemical industry, cleaning industry, detergent industry, home care industry, cosmetic industry, or food industry, preferably pet food industry.
For example, the aqueous formulation according to the first or third aspect can be added to agrochemical products such as fertilizers or plant protection products. In particular, agrochemical products can be coated with the aqueous formulation according to the first or third aspect. Alternatively, the aqueous formulation according to the first or third aspect can be added to foodstuffs or food supplements. In particular, foodstuffs or food supplements can be coated with the aqueous formulation according to the first or third aspect. The aqueous formulation can also be added to/incorporated/worked into products used in the agrochemical industry, cleaning industry, home care industry, detergent industry, cosmetic industry, or food industry.

In a sixth aspect, the present invention relates to a substrate comprising the aqueous formulation such as aqueous solution according to the first or third aspect.

The substrate may be any substrate that benefits from the aqueous formulation. It is preferred that the substrate is coated with the aqueous formulation according to the first or third aspect. The coating is a covering that is applied to the substrate, in particular to the surface of the substrate, to be coated. The coating itself may be an all-over coating, completely covering the substrate, or it may only cover parts of the substrate.
In one embodiment, the coating covers at least 1%, preferably at least 30%, more preferably at least 50%, even more preferably at least 80%, and most preferably at least 90% or even 100% of the surface of the substrate. In one preferred embodiment, the coating is an uniform and/or homogenous coating. It has preferably a thickness of between 10 nm and 1 mm and more preferably a thickness of between 50 nm and 0.5 μm.
The coating is preferably achieved by dip coating and/or spray coating.
In an example, the substrate is a plant. In this case, the coating may be applied to (parts of) the root(s) of the plant and/or sprout(s) of the plant/aboveground part(s) of the plant. The coating may also be applied to (parts of) the fruits and/or blossoms/flower petals of the plant. It should be clear that a plant, in particular (a/an) sprout(s) of a plant/aboveground part(s) of a plant, also comprise(s) leaves/leafage. Thus, the coating preferably also covers the leaves/leafage. The coating is preferably applied pre-harvest, e.g. to a seedling, a growing plant, or full-grown plant. Said substrate may be selected from the group consisting of a plant, a part of a plant (e.g. root and/or sprout), plant seeds, a nutrient, a fruit, and a flower.

In a seventh aspect, the present invention relates to a composition comprising the aqueous formulation such as aqueous solution according of the first or third aspect.

The composition may be any composition that benefits from the aqueous formulation according to the first or third aspect. The composition may be, for example, a composition useful in the agrochemical industry, cleaning industry, home care industry, detergent industry, cosmetic industry, or food industry. Thus, said composition may be a food composition, a cleaning composition, a fertilizer composition, or a plant or seed protecting composition. In these cases, the aqueous formulation may be integrated, embedded, or worked into the composition.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURE

The following figures are merely illustrative of the present invention and should not be construed to limit the scope of the invention as indicated by the appended claims in any way.

FIG. 1: Protein formulations before and after neutralization are shown (Example 1): from left to right: protein formulation before neutralization at pH 12.5, 10 M urea, and 10% protein (w/v); protein formulations after neutralization at pH 10.0, pH 8.0-8.5, and pH 7.0. The protein formulations after neutralization showed a gel character. As protein eADF4(C16) was used.

FIG. 2: Protein formulations before and after neutralization are shown (Example 2): from left to right: protein formulation before neutralization at pH 13.0, 8 M urea, and 10% protein (w/v); protein formulations after neutralization at pH 10.0, pH 8.0, and pH 7.0. The protein formulations after neutralization showed a gel character. As protein eADF4(C16) was used.

FIG. 3: Protein formulations before and after neutralization are shown (Example 3): from left to right: protein formulation before neutralization at pH 12.5, 8 M urea, and 5% protein (w/v); protein formulations after neutralization at pH 9.5, pH 8.0, and pH 7.0. The protein formulations after neutralization showed a gel character. As protein eADF4(C16) was used.

FIG. 4: Protein formulations before and after neutralization are shown (Example 4): from left to right: protein formulation before neutralization at pH 13, 10 M urea, and 12.5% protein (w/v); protein formulations after neutralization at pH 10.0, pH 8.0 and pH 5.5. The protein formulations after neutralization showed a gel character. As protein eADF4(C16) was used.

FIG. 5: Protein formulations before and after neutralization are shown (Example 5): from left to right: protein formulation before neutralization at pH 13.0, 10 M urea, and 15% protein (w/v); protein formulations after neutralization at pH 9.0, pH 8.0 and pH 6.0. The protein formulations after neutralization showed a gel character. As protein eADF4(C16) was used.

### EXAMPLES

The examples given below are for illustrative purposes only and do not limit the invention described above in any way.

### Example 1

3.00 g of silk polypeptide eADF4(C16) were added into a 50 mL tube (Sarstedt). 24.4 mL of 10 M urea solution were added. The mixture was mixed on a vortexer (VORTEX-2-GENIE) for about 30 sec. After mixing, the protein powder was homogeneously dispersed and the dispersion was a milky white, liquid dispersion with a pH of 6.0.

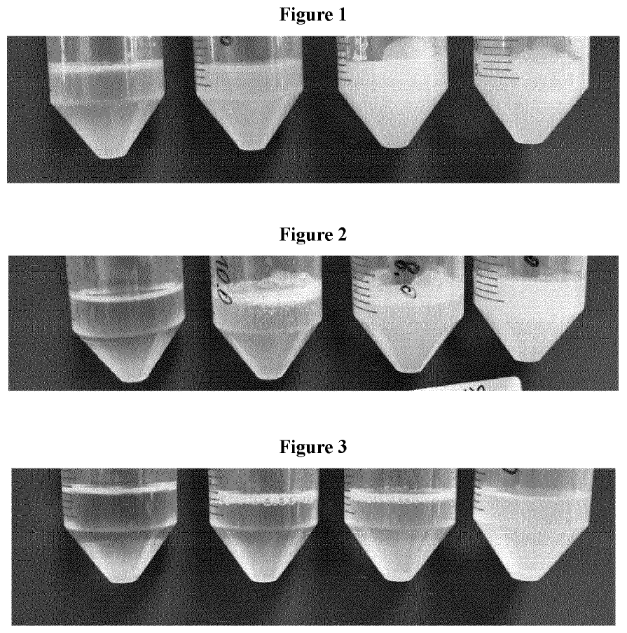

2.50 mL of 1 M sodium hydroxide solution (NaOH) were added to the aqueous protein dispersion. The protein dispersion was shaken manually immediately after addition of sodium hydroxide (NaOH) for about 20 sec and subsequently mixed on a vortexer (VORTEX-2-GENIE) for about 60 sec. After about 120 sec, the dispersion turned into a creamy aqueous formulation. Subsequently, the formulation was mixed manually with a spatula for about 5 min. After this mixing time, the formulation turned into a flowable homogeneous aqueous solution (see FIG. 1, first tube in the row). The pH of the solution was measured to pH 12.5 with pH indicator sticks (Carl Roth).

A flowable, viscous protein formulation with 10% protein, 10 M urea was produced at pH 12.5. Subsequently, the protein formulation was neutralized with 5 M hydrochloric acid to different pH. Three aliquots of 7.5 mL each were transferred to 50 mL tubes (Sarstedt). Hydrochloric acid was added to the aliquots in volumes of 0.050, 0.085 mL, and 0.120 mL and the samples were mixed manually via a spatula for about 90 sec to obtain a pH of pH 10.0, pH 8.5, and pH 7.0, respectively.

After a storage time of 5 min, the three formulations showed a gel character (see Figure, second to fourth tube in the row).

Consequently, protein formulations with a gel character with 10% protein, 10 M urea were produced at pH 7.0, pH 8.5, and pH 10.0.

### Example 2

3.00 g of silk polypeptide eADF4(C16) were added into a 50 mL tube (Sarstedt). 23.6 mL of 8 M urea solution were added. The mixture was mixed on a vortexer (VORTEX-2-GENIE) for about 30 sec. After mixing, the protein powder was homogeneously dispersed and the dispersion was a milky white, liquid dispersion with a pH of 6.0.

3.30 mL of 1 M sodium hydroxide (NaOH) solution were added to the aqueous protein dispersion. The protein dispersion was shaken manually immediately after addition of sodium hydroxide (NaOH) for about 20 sec and subsequently mixed on a vortexer (VORTEX-2-GENIE) for about 60 sec. After about 120 sec, the dispersion turned into a creamy aqueous formulation. Subsequently, the formulation was mixed manually with a spatula for about 5 min. After this mixing time, the formulation turned into a flowable homogeneous aqueous solution (see FIG. 2, first tube in the row). The pH of the solution was measured to pH 13.0 with pH indicator sticks (Carl Roth).

Consequently, a flowable, viscous protein formulation with 10% protein, 8 M urea was produced at pH 13.0.

Subsequently, the protein formulation was neutralized with 5 M hydrochloric acid to different pH. Three aliquots of 7.5 mL each were transferred to 50 mL tubes (Sarstedt). Hydrochloric acid was added to the aliquots in volumes of 0.050, 0.090 mL, and 0.110 mL and the samples were mixed manually via a spatula for about 90 sec to obtain a pH of pH 10.0, pH 8.0, and pH 7.0, respectively.

After a storage time of 5 min, the three formulations showed a gel character (see Figure, second to fourth tube in the row).

Consequently, protein formulations with a gel character with 10% protein, 8 M urea were produced at pH 7.0, pH 8.0, and pH 10.0.

### Example 3

1.50 g of silk polypeptide eADF4(C16) were added into a 50 mL tube (Sarstedt). 27.15 mL of 8 M urea solution were added. The mixture was mixed on a vortexer (VORTEX-2-GENIE) for about 30 sec. After mixing, the protein powder was homogeneously dispersed and the dispersion was a milky white, liquid dispersion with a pH of 6.5.

1.25 mL of 1 M sodium hydroxide solution (NaOH) were added to the aqueous protein dispersion. The protein dispersion was shaken manually immediately after addition of sodium hydroxide (NaOH) for about 20 sec and subsequently mixed on a vortexer (VORTEX-2-GENIE) for about 60 sec. After about 120 sec, the dispersion turned into a creamy low viscose aqueous formulation.

Subsequently, the formulation was mixed manually with a spatula for about 5 min. After this mixing time, the formulation turned into a flowable homogeneous aqueous solution (see FIG. 3, first tube in the row). The pH of the solution was measured to pH 12.5 with pH indicator sticks (Carl Roth).

Consequently, a flowable, viscous protein formulation with 5% protein, 8 M urea was produced at pH 12.5.

Subsequently, the protein formulation was neutralized with 5 M hydrochloric acid to different pH. Three aliquots of 7.5 mL each were transferred to 50 mL tubes (Sarstedt). Hydrochloric acid was added to the aliquots in volumes of 0.035, 0.055 mL, and 0.090 mL and the samples were mixed manually via a spatula for about 90 sec to obtain a pH of pH 9.5, pH 8.0, and pH 7.0, respectively.

After a storage time of 10 min, the three formulations showed a gel character (see Figure, second to fourth tube in the row).

Consequently, protein formulations with a gel character with 5% protein, 8 M urea were produced at pH 7.0, pH 8.0, and pH 9.5.

### Example 4

3.75 g of silk polypeptide eADF4(C16) were added into a 50 mL tube (Sarstedt). 21.85 mL of 10 M urea solution were added. The mixture was mixed on a vortexer (VORTEX-2-GENIE) for about 30 sec. After mixing, the protein powder was homogeneously dispersed and the dispersion was a milky white, liquid dispersion with a pH of 6.0.

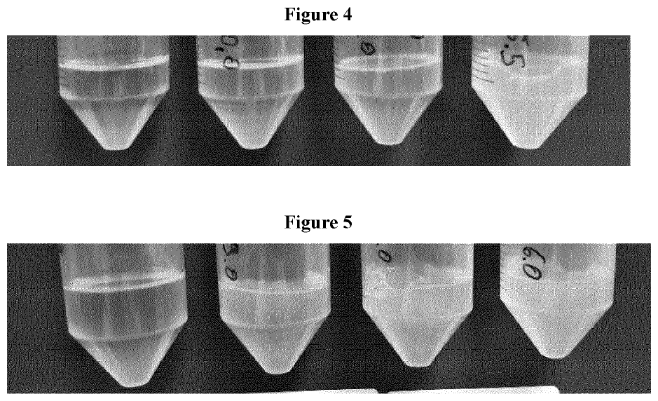

4.30 mL of 1 M sodium hydroxide (NaOH) solution were added to the aqueous protein dispersion. The protein dispersion was shaken manually immediately after addition of sodium hydroxide (NaOH) for about 20 sec and subsequently mixed on a vortexer (VORTEX-2-GENIE) for about 60 sec. After about 120 sec, the dispersion turned into a creamy aqueous formulation. Subsequently, the formulation was mixed manually with a spatula for about 5 min. After this mixing time, the formulation turned into a flowable homogeneous aqueous solution (see FIG. 4, first tube in the row). The pH of the solution was measured to pH 13.0 with pH indicator sticks (Carl Roth).

Consequently, a flowable, viscous protein formulation with 12.5% protein, 10 M urea was produced at pH 13.0.

Subsequently, the protein formulation was neutralized with 5 M hydrochloric acid to different pH. Three aliquots of 7.5 mL each were transferred to 50 mL tubes (Sarstedt). Hydrochloric acid was added to the aliquots in volumes of 0.110, 0.145 mL, and 0.160 mL and the samples were mixed manually via a spatula for about 90 sec to obtain a pH of pH 10.0, pH 8.0, and pH 5.5, respectively.

After a storage time of 5 min, the three formulations showed a gel character (see Figure, second to fourth tube in the row).

Consequently, protein formulations with a gel character with 12.5% protein, 10 M urea were produced at pH 5.5, pH 8.0, and pH 10.0.

### Example 5

4.50 g of silk polypeptide eADF4(C16) were added into a 50 mL tube (Sarstedt). 20.2 mL of 10 M urea solution were added. The mixture was mixed on a vortexer (VORTEX-2-GENIE) for about 30 sec. After mixing, the protein powder was homogeneously dispersed and the dispersion was a milky white, liquid dispersion with a pH of 5.5.

5.16 mL of 1 M sodium hydroxide solution (NaOH) were added to the aqueous protein dispersion. The protein dispersion was shaken manually immediately after addition of sodium hydroxide (NaOH) for about 20 sec and subsequently mixed on a vortexer (VORTEX-2-GENIE) for about 60 sec. After about 120 sec, the dispersion turned into a creamy aqueous formulation.

Subsequently, the formulation was mixed manually with a spatula several times. After this mixing time, the formulation turned into a flowable homogeneous aqueous solution (see Figure, first tube in the row). The pH of the solution was measured to pH 13 with pH indicator sticks (Carl Roth).

A flowable, viscous protein formulation with 15.0% protein, 10 M urea was produced at pH 13.0.

Subsequently, the protein formulation was neutralized with 5 M hydrochloric acid to different pH. Three aliquots of 7.5 mL each were transferred to 50 mL tubes (Sarstedt). Hydrochloric acid was added to the aliquots in volumes of 0.115, 0.180 mL, and 0.200 mL and the samples were mixed manually via a spatula for about 90 sec to obtain a pH of pH 9.0, pH 8.0, and pH 6.0, respectively.

After a storage time of 5 min, the three formulations showed a gel character (see Figure, second to fourth tube in the row).

Consequently, protein formulations with a gel character with 15.0% protein, 10 M urea were produced at pH 6.0, pH 8.0, and pH 9.0.

1. An aqueous formulation comprising a silk polypeptide and urea, wherein the concentration of the silk polypeptide in the formulation is in the range of 0.5% by weight to 30% by weight.

2. (canceled)

3. The aqueous formulation of claim 1, wherein the aqueous formulation is liquid, has a fibrillary or gel-like structure, or is solid, and wherein the fibrillary or gel-like structure is a hydrogel.

4. (canceled)

5. The aqueous formulation of claim 1, wherein the concentration of urea in the formulation is in the range of 3 M to 10 M.

6. The aqueous formulation of claim 1, wherein the aqueous formulation
has a pH in the range of 9 to 13 (before neutralization), or
has a pH in the range of 5.5 to 11 (after neutralization).

7. The aqueous formulation of claim 1, wherein the silk polypeptide comprises at least two identical repetitive units.

8. The aqueous formulation of claim 7, wherein the repetitive units are independently selected from the group consisting of module C (SEQ ID NO: 1) or variants thereof, module C^{Cys} (SEQ ID NO: 2) or variants thereof, and module C^{Lys} (SEQ ID NO: 7) or variants thereof.

9-10. (canceled)

11. A method for producing an aqueous formulation comprising the steps of:
(i) providing an aqueous dispersion comprising a silk polypeptide and urea, wherein the concentration of the silk polypeptide in the dispersion is in the range of 0.5% by weight to 30% by weight, and
(ii) forming an aqueous formulation from the dispersion, wherein the silk polypeptide is comprised in a solubilized form.

12. (canceled)

13. The method of claim 11, wherein the aqueous dispersion has a pH of <9.

14. The method of claim 11, wherein the aqueous formulation is formed by increasing the pH of the aqueous dispersion.

15. The method of claim 14, wherein the pH of the aqueous dispersion is increased by adding a basic buffer or a base to said aqueous dispersion.

16. (canceled)

17. The method of claim 14, wherein the pH of the aqueous dispersion is increased to a pH in the range of 9 to 13 (before neutralization).

18. The method of claim 14, wherein the method further comprises the step of reducing the pH of the aqueous formulation.

19. The method of claim 18, wherein the pH of the aqueous formulation is reduced by adding an acid buffer or an acid to said aqueous formulation.

20. (canceled)

21. The method of claim 18, wherein the pH of the aqueous formulation is reduced to a pH in the range of 5.5 to 11 (after neutralization).

22. The method of claim 11, wherein the aqueous formulation is liquid, has a fibrillary or gel-like structure, or is solid, and wherein the fibrillary or gel-like structure is a hydrogel.

23. (canceled)

24. The method of claim 11, wherein the concentration of urea in the formulation is in the range of 5 M to 10 M.

25. The method claim 11, wherein the silk polypeptide comprises at least two identical repetitive units.

26. The method of claim 25, wherein the repetitive units are independently selected from the group consisting of module C (SEQ ID NO: 1) or variants thereof, module C^{Cys} (SEQ ID NO: 2) or variants thereof, and module C^{Lys} (SEQ ID NO: 7) or variants thereof.

27-28. (canceled)

29. An aqueous formulation obtainable by the method of claim 11.

30-31. (canceled)

32. A substrate or composition comprising the aqueous formulation of claim 1.

33. (canceled)

34. A substrate or composition comprising the aqueous formulation of claim 29.
